(19) United States
(12) Patent Application Publication
(10) Pub. No.: US 2026/0242386 A1
(43) Pub. Date: Aug. 20, 2026

(54) SOLID STATE FORMS OF DIBUPRENORPHINE ETHYL ETHER AND PROCESS FOR PREPARATION THEREOF

(71) Applicant: TAPI Czech Industries s.r.o., Opava-Komarov (CZ)

(72) Inventors: Zuzana Przeczkova, Ostrava (CZ); Adela Benysek Bartova, Ostrava-Michalkovice (CZ); Pavel Kolesa, Hlubocec (CZ); Leopold Bittner, Opava (CZ); Alexandr Jegorov, Dobra Voda (CZ)

(21) Appl. No.: 18/876,703

(22) PCT Filed: Jun. 21, 2023
(86) PCT No.: PCT/IB2023/056430

§ 371 Date: Dec. 19, 2024

Related U.S. Application Data
(60) Provisional application No. 63/354,282, filed on Jun. 22, 2022.
(60) Provisional application No. 63/426,400, filed on Nov. 18, 2022.
(60) Provisional application No. 63/468,177, filed on May 22, 2023.

(51) Int. Cl. C07D 489/12; A61K 31/485

(52) U.S. Cl. CPC C07D 489/12; A61K 31/485

(57) ABSTRACT

Solid state forms of Dibuprenorphine-ethyl-ether and Dibuprenorphine-ethyl-ether 2HCl are provided, as well as processes for the preparation thereof, and pharmaceutical compositions thereof.

### FIELD OF THE DISCLOSURE

The present disclosure encompasses solid state forms of Dibuprenorphine-ethyl-ether, in embodiments crystalline polymorphs of Dibuprenorphine-ethyl-ether and Dibuprenorphine-ethyl-ether 2HCl, processes for preparation thereof, and pharmaceutical compositions thereof.

### BACKGROUND OF THE DISCLOSURE

Dibuprenorphine-ethyl-ether, 2,2′-((4aR,4a′R,6S,6'S,7S,7'S,12bR,12b′R)-9,9′-(ethane-1,2-diylbis(oxy))bis(3-(cyclopropylmethyl)-7-methoxy-1,2,3,4,5,6,7,7a-octahydro-4a, 7-ethano-4,12-methanobenzofuro[3,2-e]isoquinoline-9,6-diyl))bis(3,3-dimethylbutan-2-ol), has the following chemical structure:

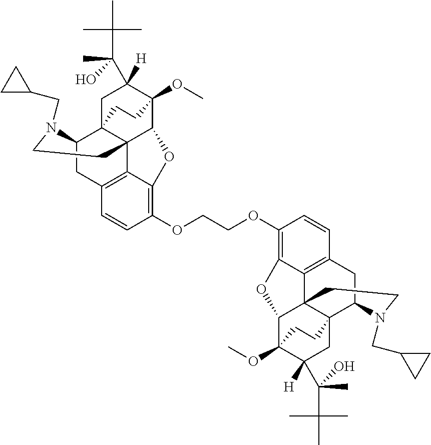 [SMILES: CO[C@@]12CC[C@@]3(C[C@@H]1[C@](C)(O)C(C)(C)C)[C@H]1Cc4ccc(OCCOc5ccc6c7c5O[C@H]5[C@]8(OC)CC[C@@]9(C[C@@H]8[C@](C)(O)C(C)(C)C)[C@@H](C6)N(CC6CC6)CC[C@]759)c5c4[C@@]3(CCN1CC1CC1)[C@H]2O5]

Dibuprenorphine-ethyl-ether is a dimer of buprenorphine, and it is developed for the treatment of Irritable Bowel Syndrome-Diarrhea (IBS-D) and pain.

The compound is described in International Publication No. WO 2015/168031.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Dibuprenorphine-ethyl-ether and Dibuprenorphine-ethyl-ether 2HCl.

### SUMMARY OF THE DISCLOSURE

The present disclosure provides crystalline polymorphs of Dibuprenorphine-ethyl-ether and Dibuprenorphine-ethyl-ether 2HCl, processes for preparation thereof, and pharmaceutical compositions thereof. These crystalline polymorphs can be used to prepare other solid state forms of Dibuprenorphine-ethyl-ether, Dibuprenorphine-ethyl-ether salts and their solid state forms.

The present disclosure also provides uses of the said solid state forms of API in the preparation of other solid state forms of Dibuprenorphine-ethyl-ether or salts thereof.

The present disclosure provides crystalline polymorphs of Dibuprenorphine-ethyl-ether or Dibuprenorphine-ethyl-ether 2HCl for use in medicine, including for the treatment of Irritable Bowel Syndrome-Diarrhea (IBS-D) or pain.

The present disclosure also encompasses the use of crystalline polymorphs of Dibuprenorphine-ethyl-ether or Dibuprenorphine-ethyl-ether 2HCl of the present disclosure for the preparation of pharmaceutical compositions and/or formulations.

In another aspect, the present disclosure provides pharmaceutical compositions comprising crystalline polymorphs of Dibuprenorphine-ethyl-ether or Dibuprenorphine-ethyl-ether 2HCl according to the present disclosure.

The present disclosure includes processes for preparing the above mentioned pharmaceutical compositions. The processes includes combining any one or a combination of the crystalline polymorphs of Dibuprenorphine-ethyl-ether or Dibuprenorphine-ethyl-ether 2HCl with at least one pharmaceutically acceptable excipient.

The crystalline polymorph of Dibuprenorphine-ethyl-ether or Dibuprenorphine-ethyl-ether 2HCl as defined herein and the pharmaceutical compositions or formulations of the crystalline polymorph of Dibuprenorphine-ethyl-ether or Dibuprenorphine-ethyl-ether 2HCl may be used as medicaments, such as for the treatment of Irritable Bowel Syndrome-Diarrhea (IBS-D) or pain.

The present disclosure also provides methods of treating Irritable Bowel Syndrome-Diarrhea (IBS-D) or pain, by administering a therapeutically effective amount of any one or a combination of the crystalline polymorphs of Dibuprenorphine-ethyl-ether or Dibuprenorphine-ethyl-ether 2HCl of the present disclosure, or at least one of the above pharmaceutical compositions, to a subject suffering from Irritable Bowel Syndrome-Diarrhea (IBS-D) or pain, or otherwise in need of the treatment.

The present disclosure also provides uses of crystalline polymorphs of Dibuprenorphine-ethyl-ether or Dibuprenorphine-ethyl-ether 2HCl of the present disclosure, or at least one of the above pharmaceutical compositions, for the manufacture of medicaments for treating e.g. Irritable Bowel Syndrome-Diarrhea (IBS-D) or pain.

### BRIEF DESCRIPTION OF THE DRAWINGS

FIG. 1 shows a characteristic X-ray powder diffraction pattern (XRPD) of Dibuprenorphine-ethyl-ether Form 1.

FIG. 2 shows a characteristic XRPD of Dibuprenorphine-ethyl-ether 2HCl, Form C1.

FIG. 3 shows a characteristic XRPD of Dibuprenorphine-ethyl-ether, amorphous.

### DETAILED DESCRIPTION OF THE DISCLOSURE

The present disclosure encompasses solid state forms of Dibuprenorphine-ethyl-ether, including crystalline polymorphs of Dibuprenorphine-ethyl-ether and Dibuprenorphine-ethyl-ether 2HCl, processes for preparation thereof, and pharmaceutical compositions thereof.

Solid state properties of Dibuprenorphine-ethyl-ether or Dibuprenorphine-ethyl-ether 2HCl and crystalline polymorphs thereof can be influenced by controlling the conditions under which Dibuprenorphine-ethyl-ether or Dibuprenorphine-ethyl-ether 2HCl and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Dibuprenorphine-ethyl-ether described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Dibuprenorphine-ethyl-ether. In some embodiments of the disclosure, the described crystalline polymorph of Dibuprenorphine-ethyl-ether may contain from about 1% to about 20% (w/w), from about 5% to about 20% (w/w), or from about 5% to about 10% (w/w) of one or more other crystalline polymorph of the same Dibuprenorphine-ethyl-ether.

Isostructural forms may be referred to herein as two or more crystalline forms of a substance, which have a common structural similarity, including approximately similar interplanar spacing in the crystal lattice. Isostructural forms have the same space group and similar or identical unit cell dimensions. Due to their structural similarity, isostructural forms typically have similar, but not necessarily identical, X-ray powder diffraction patterns. Isostructural forms may be based upon a substance that is a neutral molecule, a salt or a molecular complex. Isostructural forms may be solvates, including hydrates, and desolvated solvate crystal forms of the substance. Solvated forms of isostructural forms typically contain one or more solvents, including water, in the crystal lattice. The solvent or solvents in the crystal lattice may be the solvent or solvents of crystallization used in preparing the crystal form.

Depending on which other crystalline polymorphs a comparison is made, the crystalline polymorphs of Dibuprenorphine-ethyl-ether of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Dibuprenorphine-ethyl-ether referred to herein as being characterized by graphical data “as depicted in” or “as substantially depicted in” a Figure will thus be understood to include any crystal forms of Dibuprenorphine-ethyl-ether characterized with the graphical data having such small variations, as are well known to the skilled person, in comparison with the Figure.

A solid state form of Dibuprenorphine-ethyl-ether or a salt thereof might also be determined by the unit cell dimensions of its crystal structure. The unit cell dimensions might be determined from single crystal XRD or by structure refinement from powder XRD, such as by Rietveld or Le Bail refinement.

As used herein, and unless stated otherwise, the term “anhydrous” in relation to crystalline forms of Dibuprenorphine-ethyl-ether, relates to a crystalline form of Dibuprenorphine-ethyl-ether which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Dibuprenorphine-ethyl-ether of the present disclosure corresponds to a crystalline polymorph of Dibuprenorphine-ethyl-ether that is physically separated from the reaction mixture in which it is formed.

As used herein, unless stated otherwise, the XRPD measurements are taken using copper Kα radiation wavelength 1.5418 Å. XRPD peaks reported herein are measured using Cuk a radiation, 2=1.5418 Å, typically at a temperature of 25±3° C.

A thing, e.g., a reaction mixture, may be characterized herein as being at, or allowed to come to “room temperature,” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

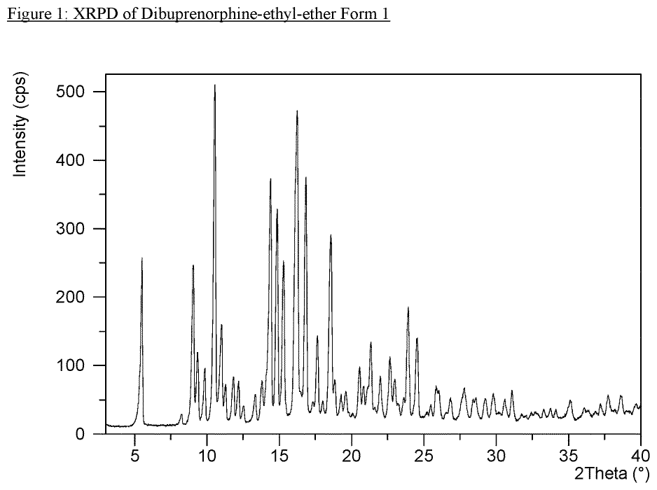

The present disclosure includes a crystalline polymorph of Dibuprenorphine-ethyl-ether, designated Form 1. The crystalline Form 1 of Dibuprenorphine-ethyl-ether may be characterized by data selected from one or more of the following: an X-ray powder diffraction pattern substantially as depicted in FIG. 1; an X-ray powder diffraction pattern having peaks at 5.5, 9.1, 10.6, 14.9 and 16.3 degrees 2-theta±0.2 degrees 2-theta; and combinations of these data.

Crystalline Form 1 of Dibuprenorphine-ethyl-ether may be further characterized by an X-ray powder diffraction pattern having peaks at 5.5, 9.1, 10.6, 14.9 and 16.3 degrees 2-theta ±0.2 degrees 2-theta, and also having any one, two, three, four or five additional peaks selected from 9.4, 11.9, 14.4, 15.3 and 16.9 degrees 2-theta±0.2 degrees 2-theta.

In one embodiment of the present disclosure, crystalline Form 1 of Dibuprenorphine-ethyl-ether is isolated.

Crystalline Form 1 of Dibuprenorphine-ethyl-ether is anhydrous form.

Crystalline Form 1 of Dibuprenorphine-ethyl-ether may be characterized by each of the above characteristics alone/or by all possible combinations, e.g., an XRPD pattern having peaks at 5.5, 9.1, 10.6, 14.9 and 16.3 degrees 2-theta±0.2 degrees 2-theta; an XRPD pattern as depicted in FIG. 1, and combinations thereof.

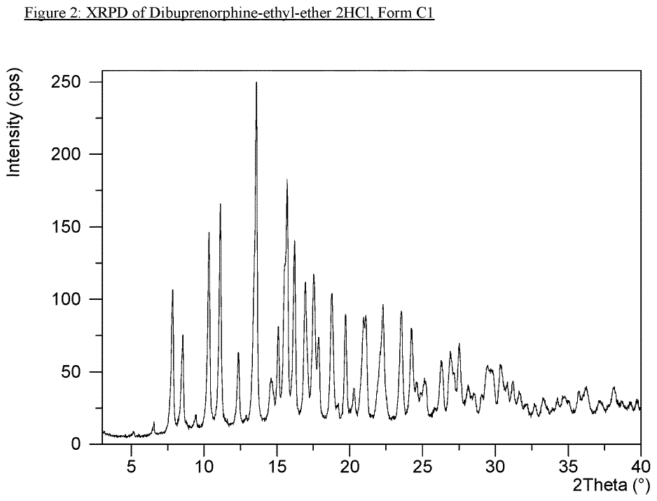

The present disclosure includes a crystalline polymorph of Dibuprenorphine-ethyl-ether 2HCl, designated Form C1. The crystalline Form C1 of Dibuprenorphine-ethyl-ether 2HCl may be characterized by data selected from one or more of the following: an X-ray powder diffraction pattern substantially as depicted in FIG. 2; an X-ray powder diffraction pattern having peaks at 7.8, 8.5, 10.3, 11.1 and 13.6 degrees 2-theta±0.2 degrees 2-theta; and combinations of these data.

Crystalline Form C1 of Dibuprenorphine-ethyl-ether 2HCl may be further characterized by an X-ray powder diffraction pattern having peaks at 7.8, 8.5, 10.3, 11.1 and 13.6 degrees 2-theta±0.2 degrees 2-theta, and also having any one, two, three, four or five additional peaks selected from 12.4, 15.1, 16.2, 17.0 and 18.8 degrees 2-theta±0.2 degrees 2-theta.

In one embodiment of the present disclosure, crystalline Form C1 of Dibuprenorphine-ethyl-ether 2HCl is isolated.

Crystalline Form C1 of Dibuprenorphine-ethyl-ether 2HCl may be characterized by each of the above characteristics alone/or by all possible combinations, e.g., an XRPD pattern having peaks at 7.8, 8.5, 10.3, 11.1 and 13.6 degrees 2-theta±0.2 degrees 2-theta; an XRPD pattern as depicted in FIG. 2, and combinations thereof.

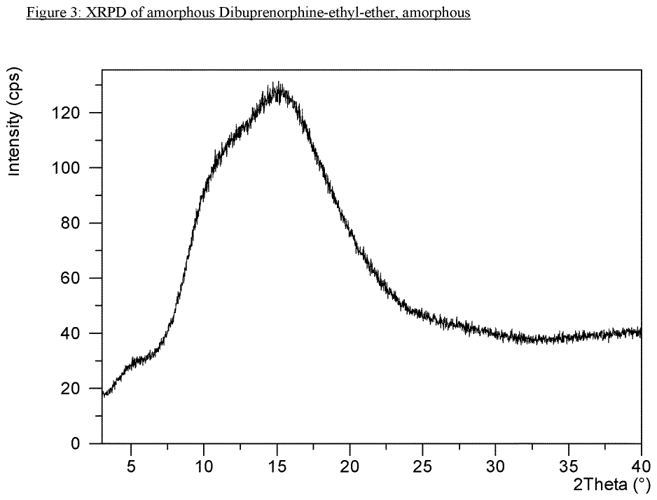

The present disclosure includes an amorphous form of Dibuprenorphine-ethyl-ether. The amorphous Form of Dibuprenorphine-ethyl-ether may be characterized by an X-ray powder diffraction pattern substantially as depicted in FIG. 3.

In one embodiment of the present disclosure, amorphous Form of Dibuprenorphine-ethyl-ether is isolated.

The present disclosure further includes isostructural forms of solvated Dibuprenorphine-ethyl-ether 2HCl of the general formula:

$$DBP \times 2 HCl \times [ {H}_{2} O ] n \times [ S ]$$

### DBP: Dibuprenorphine-Ethyl-Ether

2 HCl: di-hydrochloride
n=0-3
S: water-miscible or water-immiscible organic solvent
The isostructural forms of solvated Dibuprenorphine-ethyl-ether 2HCl may be anhydrous or hydrated forms, e.g. Dibuprenorphine-ethyl-ether 2HCl solvate-hydrates.

The isostructural forms of the present disclosure can be characterized by the monoclinic space group P2₁, having average unit cell parameters of

$$a = 13.6 Å to 1.77 Å$$

$$b = 13. Å to 13.2 Å$$

$$c = 13.6 Å to 17.5 Å$$

$$α = γ = 90 °$$

$$β = 95 ° to 102 °$$

$$unit cell volume = 2950 Å 3 to 3117 Å 3$$

wherein a, b and c represent the crystal axes lengths, and a, B and y represent the angels between the crystal axes.

The unit cell parameters of the isostructural forms of the present disclosure were obtained from Rietveld refinement based on single crystal structure data of Dibuprenorphine-ethyl-ether 2HCl Form C5.

The isostructural forms of the present invention can be prepared by crystallization from a solvent. Typical solvents of crystallization include water and all classes of organic and other types of laboratory solvents, including, but not limited to: alcohols, such as methanol, ethanol, n-propanol, isopropanol, n-butanol, sec-butanol, t-butanol, phenol, glycerol, and the like; carbonyl-containing solvents, such as acetone, methyl ethyl ketone, formic acid, acetic acid, ethyl acetate, butyl acetate, N,N-dimethylformamide, and the like; hydrocarbons, such as pentane, hexane, cyclohexane, benzene, toluene, xylenes, and the like; halogenated solvents, such as dichloromethane, chloroform, carbon tetrachloride, and the like; and laboratory solvents containing other heteroatoms and/or functional groups, such as acetonitrile, tetrahydrofuran, diethyl ether, diisopropyl ether, carbon disulfide, dimethyl sulfoxide, 1,4-dioxane, nitrobenzene, nitromethane, pyridine, and the like.

Crystalline Form C1 of Dibuprenorphine-ethyl-ether 2HCl of the present disclosure can also be characterized by the following unit cell dimensions:

| Unit cell parameter | Rietveld refinement 293K | |
|---|---|---|
| cell_length_a | 17,238(6) | Å |
| cell_length_b | 13,027(8) | Å |
| cell_length_c | 13,604(9) | Å |
| cell_angle_alpha | 90° | |
| cell_angle_beta | 96,752(2)° | |
| cell_angle_gamma | 90° | |
| cell_volume | 3034.2(1) | Å³ |

| | |
|---|---|
| symmetry_cell_setting | monoclinic |
| Symmetry space group name | P 2₁ |

The crystalline Form C1 of Dibuprenorphine-ethyl-ether 2HCl is an ethyl acetate solvate. The crystalline Form C1 of Dibuprenorphine-ethyl-ether 2HCl may be an ethyl acetate solvate-hydrate.

The present disclosure also includes isostructural Form C2 of Dibuprenorphine-ethyl-ether 2HCl. Form C2 was prepared from iso-butyl acetate and can be characterized by the following unit cell parameters:

| Unit cell parameter | Rietveld refinement 293K | |
|---|---|---|
| cell_length_a | 17,692 | Å |
| cell_length_b | 13,019(9) | Å |
| cell_length_c | 13,831(5) | Å |
| cell_angle_alpha | 90° | |
| cell_angle_beta | 101,636(9)° | |
| cell_angle_gamma | 90° | |
| cell_volume | 3120.5(7) | Å³ |

| | |
|---|---|
| symmetry_cell_setting | monoclinic |
| Symmetry space group name | P 2₁ |

The crystalline Form C2 of Dibuprenorphine-ethyl-ether 2HCl is an iso-butyl acetate solvate. The crystalline Form C2 of Dibuprenorphine-ethyl-ether 2HCl may be an iso-butyl acetate solvate-hydrate.

The present disclosure also includes isostructural Form C3 of Dibuprenorphine-ethyl-ether 2HCl. Form C3 was prepared from ethanol and can be characterized by the following unit cell parameters

| Unit cell parameter | Rietveld refinement 293K | |
|---|---|---|
| cell_length_a | 13.641 | Å |
| cell_length_b | 13.080 | Å |
| cell_length_c | 16.958 | Å |
| cell_angle_alpha | 90° | |
| cell_angle_beta | 95.80° | |
| cell_angle_gamma | 90° | |
| cell_volume | 3010.23 | Å³ |

| | |
|---|---|
| symmetry_cell_setting | monoclinic |
| Symmetry space group name | P 2₁ |

The crystalline Form C3 of Dibuprenorphine-ethyl-ether 2HCl is an ethanol solvate. The crystalline Form C3 of Dibuprenorphine-ethyl-ether 2HCl may be an ethanol solvate-hydrate.

The present disclosure also includes isostructural Form C4 of Dibuprenorphine-ethyl-ether 2HCl. Form C4 was prepared from THE and can be characterized by the following unit cell parameters

| Unit cell parameter | Rietveld refinement 293K | |
|---|---|---|
| cell_length_a | 13.749 | Å |
| cell_length_b | 13.066 | Å |
| cell_length_c | 17.498 | Å |
| cell_angle_alpha | 90° | |
| cell_angle_beta | 100.78° | |
| cell_angle_gamma | 90° | |
| cell_volume | 3087.95 | Å³ |

| | |
|---|---|
| symmetry_cell_setting | monoclinic |
| Symmetry space group name | P 2₁ |

The crystalline Form C4 of Dibuprenorphine-ethyl-ether 2HCl is a THF solvate. The crystalline Form C4 of Dibuprenorphine-ethyl-ether 2HCl may be a THF solvate-hydrate.

The present disclosure also includes isostructural Form C5 of Dibuprenorphine-ethyl-ether 2HCl. Form C5 was prepared from acetonitrile and can be characterized by the following unit cell parameters determined from single crystal data:

| Unit cell parameter | Rietveld refinement 293K | |
|---|---|---|
| cell_length_a | 16.969 | Å |
| cell_length_b | 13.165 | Å |
| cell_length_c | 13.699 | Å |
| cell_angle_alpha | 90° | |
| cell_angle_beta | 96.03° | |
| cell_angle_gamma | 90° | |
| cell_volume | 2950.2(3) | Å³ |

| | |
|---|---|
| symmetry_cell_setting | monoclinic |
| Symmetry space group name | P 2₁ |

The crystalline Form C5 of Dibuprenorphine-ethyl-ether 2HCl is an acetonitrile solvate. The crystalline Form C5 of Dibuprenorphine-ethyl-ether 2HCl may be an acetonitrile solvate-hydrate.

The present disclosure also includes isostructural Form C6 of Dibuprenorphine-ethyl-ether 2HCl. Form C6 was prepared from acetone and can be characterized by the following unit cell parameters:

| Unit cell parameter | Rietveld refinement 293K | |
|---|---|---|
| cell_length_a | 13.707 | Å |
| cell_length_b | 13.054 | Å |
| cell_length_c | 17.426 | Å |
| cell_angle_alpha | 90° | |
| cell_angle_beta | 99.84° | |
| cell_angle_gamma | 90° | |
| cell_volume | 3072.31 | Å³ |

| | |
|---|---|
| symmetry_cell_setting | monoclinic |
| Symmetry space group name | P 2₁ |

The crystalline Form C6 of Dibuprenorphine-ethyl-ether 2HCl is an acetone solvate. The crystalline Form C6 of Dibuprenorphine-ethyl-ether 2HCl may be an acetone solvate-hydrate.

The present disclosure also includes isostructural Form C7 of Dibuprenorphine-ethyl-ether 2HCl. Form C7 was prepared from isopropanol and can be characterized by the following unit cell parameters:

| Unit cell parameter | Rietveld refinement 293K | |
|---|---|---|
| cell_length_a | 13.845 | Å |
| cell_length_b | 13.194 | Å |
| cell_length_c | 16.752 | Å |
| cell_angle_alpha | 90° | |
| cell_angle_beta | 95.53° | |
| cell_angle_gamma | 90° | |
| cell_volume | 3045.85 | Å³ |

| | |
|---|---|
| symmetry_cell_setting | monoclinic |
| Symmetry space group name | P 2₁ |

The crystalline Form C7 of Dibuprenorphine-ethyl-ether 2HCl is an isopropanol solvate. The crystalline Form C7 of Dibuprenorphine-ethyl-ether 2HCl may be an isopropanol solvate-hydrate.

The present disclosure also includes isostructural Form C8 of Dibuprenorphine-ethyl-ether 2HCl. Form C8 was prepared from n-butanol and can be characterized by the following unit cell parameters:

| Unit cell parameter | Rietveld refinement 293K | |
|---|---|---|
| cell_length_a | 13.636 | Å |
| cell_length_b | 13.084 | Å |
| cell_length_c | 17.378 | Å |
| cell_angle_alpha | 90° | |
| cell_angle_beta | 99.30° | |
| cell_angle_gamma | 90° | |
| cell_volume | 3059.63 | Å³ |

| | |
|---|---|
| symmetry_cell_setting | monoclinic |
| Symmetry space group name | P 2₁ |

The crystalline Form C8 of Dibuprenorphine-ethyl-ether 2HCl is an n-butanol solvate. The crystalline Form C8 of Dibuprenorphine-ethyl-ether 2HCl may be n-butanol solvate-hydrate.

The present disclosure also includes isostructural Form C9 of Dibuprenorphine-ethyl-ether 2HCl. Form C9 was prepared from methyl-ethyl-ketone (MEK) and can be characterized by the following unit cell parameters:

| Unit cell parameter | Rietveld refinement 293K | |
|---|---|---|
| cell_length_a | 13.666 | Å |
| cell_length_b | 13.041 | Å |
| cell_length_c | 17.093 | Å |
| cell_angle_alpha | 90° | |
| cell_angle_beta | 96.24° | |
| cell_angle_gamma | 90° | |
| cell_volume | 3028.27 | Å³ |

| | |
|---|---|
| symmetry_cell_setting | monoclinic |
| Symmetry space group name | P 2₁ |

The crystalline Form C9 of Dibuprenorphine-ethyl-ether 2HCl is a methyl-ethyl-ketone solvate. The crystalline Form C9 of Dibuprenorphine-ethyl-ether 2HCl may be a methyl-ethyl-ketone solvate-hydrate.

The above polymorphs can be used to prepare other crystalline polymorphs of Dibuprenorphine-ethyl-ether, Dibuprenorphine-ethyl-ether salts and their solid state forms.

The present disclosure encompasses a process for preparing other solid state forms of Dibuprenorphine-ethyl-ether, Dibuprenorphine-ethyl-ether salts and their solid state forms thereof. The process includes preparing any one of the Dibuprenorphine-ethyl-ether and solid state forms of Dibuprenorphine-ethyl-ether by the processes of the present disclosure, and converting that salt to said other Dibuprenorphine-ethyl-ether salt. The conversion can be done, for example, by a process including acidifying any one or a combination of the above described Dibuprenorphine-ethyl-ether and/or solid state forms thereof, and reacting the obtained Dibuprenorphine-ethyl-ether with an appropriate acid, to obtain the corresponding salt.

The present disclosure provides the above described crystalline polymorphs of Dibuprenorphine-ethyl-ether for use in the preparation of pharmaceutical compositions comprising Dibuprenorphine-ethyl-ether and/or crystalline polymorphs thereof.

The present disclosure also encompasses the use of crystalline polymorphs of Dibuprenorphine-ethyl-ether of the present disclosure for the preparation of pharmaceutical compositions of crystalline polymorph Dibuprenorphine-ethyl-ether and/or crystalline polymorphs thereof.

The present disclosure includes processes for preparing the above mentioned pharmaceutical compositions. The processes includes combining any one or a combination of the crystalline polymorphs of Dibuprenorphine-ethyl-ether of the present disclosure with at least one pharmaceutically acceptable excipient.

Pharmaceutical combinations or formulations of the present disclosure contain any one or a combination of the solid state forms of Dibuprenorphine-ethyl-ether of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Dibuprenorphine-ethyl-ether and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Dibuprenorphine-ethyl-ether can be administered. Dibuprenorphine-ethyl-ether may be formulated for administration to a mammal, in embodiments to a human, by injection. Dibuprenorphine-ethyl-ether can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Dibuprenorphine-ethyl-ether and the pharmaceutical compositions and/or formulations of Dibuprenorphine-ethyl-ether of the present disclosure can be used as medicaments, in embodiments in the treatment of Irritable Bowel Syndrome-Diarrhea (IBS-D) or pain.

The present disclosure also provides methods of treating Irritable Bowel Syndrome-Diarrhea (IBS-D) or pain by administering a therapeutically effective amount of any one or a combination of the crystalline polymorphs of Dibuprenorphine-ethyl-ether of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Powder X-ray Diffraction was performed on an X-Ray powder diffractometer PanAlytical X′pert Pro; CuKα radiation (λ=1.54187 Å); X'Celerator detector with active length 2.122 degrees 2-theta; laboratory temperature 25±3° C.; zero background sample holders. Prior to analysis, the samples were gently ground using a mortar and pestle to obtain a fine powder. The ground sample was adjusted into a cavity of the sample holder and the surface of the sample was smoothed using a cover glass.

Measurement parameters:
Scan range: 3-40 degrees 2-theta
Scan mode: continuous
Step size: 0.0167 degrees
Step size: 42 s
Sample spin: 60 rpm
Sample holder: zero background silicon plate

### Single Crystal X-Ray Diffraction Method

Data were collected on a Bruker D8 Venture Photon CMOS detector, equipped with a multilayer monochromator and a Mo Kα Incoatec microfocus sealed tube (λ=0.71073 Å) using combined q and @ scans at 180 K.

Data collection: Apex4 (Bruker, 2021); cell refinement: Apex4; data reduction: Apex4; program used to solve structure: Superflip (Palatinus & Chapuis, 2007); program used to refine structure and absolute configuration analysis: CRYSTALS (Betteridge et al., 2003); and void calculation was done by Platon (Spek, 2003).

### EXAMPLES

### Preparation of Starting Materials

Dibuprenorphine-ethyl-ether can be prepared according to methods known from the literature, for example WO 2015168031.

### Example 1: Preparation of Dibuprenorphine-Ethyl-Ether Form 1

Dibuprenorphine ethyl ether (80 mg) was dissolved in heptane (4 ml) by heating to 90° C. Clear solution was allowed to cool spontaneously to room temperature. Obtained slurry was stirred for 1 hour, filtered and dried under vacuum for 15 minutes. Prepared material was analyzed by XRPD.

### Example 2: Preparation of Dibuprenorphine-Ethyl-Ether Form 1

Dibuprenorphine ethyl ether (80 mg) was dissolved in dimethylformamide (2 ml) by heating to 80° C. Clear solution was allowed to cool spontaneously to room temperature. Obtained slurry was stirred for 1 hour, filtered and dried under vacuum for 15 minutes. Prepared material was analyzed by XRPD.

### Example 3: Preparation of Dibuprenorphine-Ethyl-Ether Form 1

Dibuprenorphine ethyl ether (700 mg) was dissolved in THF (10 ml) at room temperature. Clear solution was evaporated to dryness on rotavapor. Obtained material was analyzed by XRPD.

### Example 4: Preparation of Dibuprenorphine-Ethyl-Ether 2HCl, Form C1

Dibuprenorphine ethyl ether (base, Form 1, 4.4 grams) was suspended in ethyl acetate (66 ml) at room temperature. Hydrochloric acid (35%, 2.1 mol eq.) was added dropwise at room temperature. The suspension was stirred at room temperature for about 3 hours, the solid was filtered and dried under vacuum for about 60 minutes and then at 50° C. for 6 hours under nitrogen flow. The solid was analyzed by XRPD; Dibuprenorphine-ethyl-ether 2HCl, Form C1 was obtained.

### Example 5: Preparation of Amorphous Dibuprenorphine-Ethyl-Ether

Dibuprenorphine ethyl ether (base, 5 grams) was dissolved in 1,4-dioxane (250 ml) at elevated temperature (around 45° C.). A clear solution was obtained, cooled to room temperature and frozen by liquid nitrogen. Solid matter was lyophilized. Then, the solid was analyzed by XRPD; amorphous Dibuprenorphine-ethyl-ether was obtained.

### Example 6: Preparation of Dibuprenorphine-Ethyl-Ether 2HCl, Form C2

Dibuprenorphine ethyl ether (base, Form 1, 2.4 g) was suspended in iso-butyl acetate (50 mL). The suspension was heated to 50° C. during a period of about 20 minutes to obtain a clear solution. Hydrochloric acid (35%, 2.1 mol eq.) was added dropwise. The suspension was stirred at temperature of about 50° C. for about 30 minutes, then cooled to room temperature during a period of about 1 hour and finally stirred for about 1.5 hours. The solid was filtered and dried under vacuum and nitrogen flow for about 2.5 hours. The solid was analyzed by XRPD; Form C2 was obtained.

### Example 7: Preparation of Dibuprenorphine-Ethyl-Ether 2HCl, Form C3

Dibuprenorphine ethyl ether dihydrochloride (Form C1, 80 mg) was dissolved in ethanol (1 mL) at room temperature. Heptane (3 mL) was added dropwise and the obtained suspension was stirred for about 3 hours at room temperature, filtered and dried under vacuum for 60 minutes. The solid was analyzed by XRPD; Form C3 was obtained.

### Example 8: Preparation of Dibuprenorphine-Ethyl-Ether 2HCl, Form C4

Dibuprenorphine ethyl ether dihydrochloride (Form C1, 80 mg) was suspended in THF (1 mL) at room temperature. The suspension was heated to 50° C. and slurried at 50° C. for about 30 minutes, then cooled to room temperature, filtered and dried under vacuum for 20 minutes. The solid was analyzed by XRPD; Form C4 was obtained.

### Example 9: Preparation of Dibuprenorphine-Ethyl-Ether 2HCl, Form C5-Single Crystal

Dibuprenorphine ethyl ether (20 mg) was dissolved in acetonitrile (0.3 mL) at elevated temperature. The clear solution was allowed to cool spontaneously to room temperature. The solvent was very slowly evaporated and crystals were formed within 3 days. Platelet shaped single crystal was directly mounted to the goniometer head. Form C5 was obtained.

### Example 10: Preparation of Dibuprenorphine-Ethyl-Ether 2HCl, Form C6

Dibuprenorphine ethyl ether (base, Form 1, 100 mg) was suspended in acetone (1 mL) at room temperature and hydrochloric acid (35%, 2.1 mol eq.) was added dropwise. The suspension was heated to 50° C. and acetone was added up to 5 mL The suspension was slurried at 50° C. for about 30 minutes, then cooled to room temperature, stirred for 1 hour, filtered and dried under vacuum for 20 minutes. The solid was analyzed by XRPD; Form C6 was obtained.

### Example 11: Preparation of Dibuprenorphine-Ethyl-Ether 2HCl, Form C7

Dibuprenorphine ethyl ether dihydrochloride (Form C1, 80 mg) was suspended in isopropanol (1 mL) at room temperature. The suspension was heated to about 40° C. and the clear solution was stored at room temperature until crystallization. The obtained solid was filtered and analyzed by XRPD; Form C7 was obtained.

### Example 12: Preparation of Dibuprenorphine-Ethyl-Ether 2HCl, Form C8

Dibuprenorphine ethyl ether dihydrochloride (Form C1, 80 mg) was suspended in n-Butanol (1 mL) at room temperature. The suspension was heated to about 50° C. and the clear solution was stored at room temperature until crystallization. The obtained solid was filtered and analyzed by XRPD; Form C8 was obtained.

### Example 13: Preparation of Dibuprenorphine-Ethyl-Ether 2HCl, Form C9

Dibuprenorphine ethyl ether dihydrochloride (Form C1, 80 mg) was suspended in MEK (1 mL) at room temperature. The suspension was heated to about 95° C. during a period of about 140 minutes, slurried for 15 minutes and cooled to room temperature during a period of about 90 minutes. The suspension was filtered, dried under vacuum for about 15 minutes and analyzed by XRPD; Form C9 was obtained.

### Example 14: Preparation of Dibuprenorphine-Ethyl-Ether Form 1

Dibuprenorphine ethyl ether (24 grams, Form 1) was dissolved in 36 vol. of acetone under reflux. Clear solution was distilled under vacuum until obtain suspension, then suspension was stirred at about 45° C. for about 30 minutes and cooled to about 5° C. during a period of about 2 hours and stirred overnight at about 5° C. Obtained suspension was isolated, dried under vacuum and nitrogen flow to constant weight and analyzed by XRPD. Form 1 was obtained in the yield of 44% and 98.30% purity.

### Example 15: Preparation of Dibuprenorphine-Ethyl-Ether 2HCl Form C5

Dibuprenorphine ethyl ether 2HCl (80 mg, Form C1) was dissolved in acetonitrile (0.5 ml) by heating to about 75° C. during a period of about 1 hour. Clear solution was cooled to about 20° C. during a period of about 1 hour, stirred at about 20° C. for about 15 minutes. Obtained suspension was isolated and dried under vacuum. Obtained solid matter was analyzed by XRPD and Form C5 was obtained.

1. Crystalline Form I of Dibuprenorphine-ethyl-ether, which is characterized by data selected from one or more of the following:
i. an XRPD pattern having peaks at 5.5, 9.1, 10.6, 14.9 and 16.3 degrees 2-theta±0.2 degrees 2-theta; or
ii. an XRPD pattern as depicted in FIG. 1; or combinations of (i)-(ii).

2. The crystalline form of Dibuprenorphine-ethyl-ether according to claim 1, which is characterized by an XRPD pattern having peaks at 5.5, 9.1, 10.6, 14.9 and 16.3 degrees 2-theta±0.2 degrees 2-theta, and also having one, two, three, four or five additional peaks selected from 9.4, 11.9, 14.4, 15.3 and 16.9 degrees 2-theta±0.2 degrees 2-theta.

3-36. (canceled)

37. A pharmaceutical composition comprising the crystalline form according to claim 1.

38. (canceled)

39. A pharmaceutical formulation comprising the crystalline form according to claim 1 and at least one pharmaceutically acceptable excipient.

40. A method of treating a disease or condition in a subject, the method comprising administering to the subject a therapeutically effective amount of the crystalline form according to claim 1 to treat the disease or condition.

41. The method according to claim 40, wherein the disease or condition is Irritable Bowel Syndrome-Diarrhea (IBS-D) or pain.
